(19) United States
(12) Patent Application Publication
(10) Pub. No.: US 2024/0363254 A1
(43) Pub. Date: Oct. 31, 2024

(54) METHOD FOR CHARACTERIZING A TUMOUR

(71) Applicants: CENTRE HOSPITALIER UNIVERSITAIRE DE MONTPELLIER, Montpellier (FR); UNIVERSITÉ DE MONTPELLIER, Montpellier (FR); CENTRE NATIONAL DE LA RECHERCHE SCIENTIFIQUE, Paris (FR); INSTITUT NATIONAL DE LA SANTÉ ET DE LA RECHERCHE MÉDICALE, Paris (FR)

(72) Inventors: Eric RIVALS, Montpellier (FR); Christophe HIRTZ, Montpellier (FR); Alexandre DAVID, Les Matelles (FR); Sébastien RELLIER, Montpellier (FR); Luc BAUCHET, Clapiers (FR)

(21) Appl. No.: 18/292,155

(22) PCT Filed: Jul. 21, 2022
(86) PCT No.: PCT/EP2022/070551

§ 371 Date: Jan. 25, 2024

(30) Foreign Application Priority Data
Jul. 29, 2021 (FR) FR2108279

(51) Int. Cl. G16H 50/50; C12N 15/10; G01N 30/02; G01N 30/72; G01N 30/88; G01N 33/574; G06N 3/09; G16H 10/60

(52) U.S. Cl. CPC G16H 50/50; C12N 15/1003; G01N 30/7233; G01N 30/88; G01N 33/57419; G06N 3/09; G16H 10/60; G01N 2030/027; G01N 2030/8827

(57) ABSTRACT

An in vitro method is disclosed for characterizing a tumour, based on the quantitative analysis of modified and unmodified nucleosides from total cellular RNA, from extracellular RNA and/or from isolated nucleosides, extracted from a biological sample. More particularly, the invention relates to a method for predicting the grade of a glial tumour. More particularly, the invention also relates to a method for detecting a tumour. The present invention therefore lies in the fields of cancerology and molecular biology, more particularly applied to medical diagnosis.

### FIELD OF THE INVENTION

The present invention relates to an in vitro method for characterizing a tumour, based on the quantitative analysis of modified and unmodified nucleosides isolated from a biological sample. More particularly, the invention relates to a method for predicting the grade of a glial tumour. According to another particular aspect, the invention relates to a method for detecting the presence of a tumour.

The present invention is thus situated within the fields of oncology and molecular biology more particularly applied to medical diagnosis.

### STATE OF THE ART

Characterizing a tumour is an essential precondition for choosing the most suitable treatment for the patient. By “characterizing a tumour” is meant characterizing the stage or grade of development of a given tumour; it may involve in particular for example assessing the stage of development of a tumour of a known tissue, assigning a predefined grade to a tumour, or any other characterization such as in particular determining the character, initial or metastatic, of a tumour.

Gliomas, or glial tumours, are the most common tumours of the central nervous system; they are characterized by a significant variability in the age of appearance, classification, histological characteristics and capacity to progress and possibly to metastasize.

Gliomas are classified according to their morphology and their malignancy grade. The widely accepted World Health Organization (WHO) classification attributes a malignancy grade from I to IV to gliomas; glioblastomas, or grade IV tumours, being the form that is most aggressive and has the highest mortality.

One of the main limitations in the care of gliomas and glioblastomas is associated with the current lack of effective diagnostic strategies. Selecting a personalized treatment requires a precise classification of the tumours. Currently, the main diagnostic methods used clinically for detecting gliomas rely on neurological tests and neuroimaging techniques, carried out when the disease is already at an advanced stage.

Diagnosing the tumour requires an analysis of the patient's tissues originating from a biopsy or a surgical resection. Based on this sample, several molecular analyses are carried out: test of the expression of candidate genes, counting the number of copies of DNA, methylation profile, phosphoprotein pathway profiling and genetic sequencing. However, diagnoses based on biopsy have limitations in relation to determining the grade of the tumours and patient stratification. In fact, with regard for example more particularly to glial tumours, the grades of the glioma are difficult to distinguish, and more particularly grades II and III. Establishing the grade requires a difficult anatomopathological analysis, often carried out independently by two specialists. Grade II denotes a benign tumour, while grade III represents a transition towards glioblastoma multiforme, which is the most aggressive condition.

Purely histological classifications are difficult to reproduce; they are based on a visual expertise and need the intervention of two specialists. Anatomopathological analysis in tandem with image analysis by magnetic resonance imaging (MRI) is expensive and lengthy; it is dependent in particular on the waiting time to access MRI. Currently, no biomarker is sufficient on its own to guide anti-cancer treatment decisions.

A general need therefore exists for an in vitro method for characterizing a tumour, said method being objective, precise, reproducible, easy and capable of being performed if possible at an early stage of the disease. Said method would make it possible to strengthen diagnosis and facilitate patient stratification.

The publication by Janzer (“Neuropathologie et pathologie moléculaire des gliomes.” [Neuropathology and molecular pathology of gliomas] R-C Janzer, Rev. Med. Suisse, 5, 1501-4, 2009) describes the classification of gliomas according to the WHO, based on histological and immuno-histochemical criteria, as well as on genetic profiles revealing cell DNA alteration: determination of MGMT gene promoter hypermethylation (for glioblastomas) and detection of losses of chromosomes 1p and 19q (for oligodendroglial tumours).

The publication by Relier et al (“FTO-mediated cytoplasmic m⁶Aₘ demethylation adjusts stem-like properties in colorectal cancer cell.”, Nat. Commun 12, 1716, 2021) describes the regulation in the cytoplasm of the level of m⁶Aₘ methylation by the fat mass and obesity-associated protein (FTO) in cancer stem cell lines. The authors highlight the biological function of m⁶Aₘ modification and its potential adverse consequences for colorectal cancer management. This document mentions a step of analysis of fragmented mRNA by mass spectrometry (LC-MS/MS). Only the m⁶A, A, m⁶Aₘ and Aₘ nucleosides are detected and quantified.

International application WO 2007/008647 “Diagnosing and grading gliomas using a proteomics approach” relates to a method of diagnosing and grading gliomas using a proteomic approach. In this method, a tumoral tissue is analyzed by mass spectrometry and a profile of the proteins expressed is obtained.

A particular need therefore exists for an in vitro method for assessing the malignancy grade of a glial tumour, in particular its classification. A need exists in particular for an objective method making it possible to distinguish between grade II and grade III of glial tumours, to strengthen diagnosis and facilitate patient stratification.

Furthermore, a particular need exists for a method for detecting the presence of a tumour from the earliest stages. Indeed, early care for most cancers considerably increases patient survival or even makes their recovery possible. A need therefore exists for an objective method allowing early characterization of the presence of a tumour.

### DISCLOSURE OF THE INVENTION

The inventors have now developed a method for characterizing a tumour that makes use of the quantitative data of the epitranscriptome.

The epitranscriptome encompasses all of the chemical modifications borne by the ribonucleic acid (RNA) bases, a set also known by the terms “RNA epigenetics”. A method according to the invention comprises providing a biological sample from a subject suffering from a tumour and obtaining quantities of modified and unmodified nucleosides originating from said sample; said quantities are grouped together in a vector (in the mathematical sense of the term). According to a particular aspect, a method according to the invention includes the subsequent computer analysis of said vector for the characterization of a tumour. Said characterization of a tumour makes it possible to predict items of clinical and medical information on the tumour based on the sample subject to the analysis. More particularly, a method according to the invention includes the computer analysis of said vector for predicting the grade of said tumour.

For the sake of simplicity, for a given sample, the vector that groups together the quantities of each nucleoside, modified or unmodified, will be called “epitranscriptomic profile” or quite simply “profile”.

The modified and unmodified nucleosides originate from: i) total RNA extracted from cells of a biological sample from a patient, ii) extracellular RNA originating from a biological sample from a patient, and/or iii) an extract of metabolites from a biological sample isolated from a patient.

The nucleosides originating from total RNA extracted from cells of a biological sample from a patient and/or from extracellular RNA originating from a biological sample from a patient are obtained by fragmentation of the RNA into nucleotides then their dephosphorylation. The nucleosides originating from an extract of metabolites from a biological sample isolated from a patient are obtained by an extraction of the metabolites from a biological sample then the dephosphorylation of said metabolites, according to suitable methods well known to a person skilled in the art. Said metabolites originate in particular from the catabolism of RNA; the nucleosides present in monomeric form can also be denoted by the expression known as “free” nucleosides.

More particularly, the modified and unmodified nucleosides are the nucleosides present in total RNA extracted from cells of a biopsy of said tumour.

By “nucleosides” is meant the glycosylamines constituted by a nucleotide base attached to the anomeric carbon of a pentose residue by a glycosidic bond from the N1 nitrogen of a pyrimidine or the N9 of a purine. According to a particular aspect of a method according to the invention, when said pentose is ribose, the term “nucleosides” in this case denotes ribonucleosides.

In addition, the modified RNA nucleosides are denoted by the terms “epitranscriptomic marks” or “epitranscriptomic modifications”. Apart from the usual RNA nucleosides (Table 1) that can be used for characterizing tumours, modified nucleosides that can be used in a method according to the invention, in particular in the analysis of gliomas, are listed (Table 2).

TABLE 1

| | Chemical |
|---|---|
| Nucleoside | name |
| A | adenosine |
| C | cytidine |
| G | guanosine |
| U | uridine |

TABLE 2

| Nucleoside | Chemical name | Nucleoside of origin |
|---|---|---|
| Am | 2′-O-methyladenosine | A |
| m1A | 1-methyladenosine | A |
| m66A | N6,N6-dimethyladenosine | A |
| m66Am | N6,N6,2′-O-trimethyladenosine | A |
| m6A | N6-methyladenosine | A |
| m6Am | N6,2′-O-dimethyladenosine | A |
| ac4C | N4-acetylcytidine | C |
| Cm | 2′-O-methylcytidine | C |
| hm5C | 5-hydroxymethylcytidine | C |
| m3C | 3-methylcytidine | C |
| m5C | 5-methylcytidine | C |
| Gm | 2′-O-methylguanosine | G |
| m1G | 1-methylguanosine | G |
| m227G | N2,N2,7-trimethylguanosine | G |
| m27G | N2,7-dimethylguanosine | G |
| m7G | 7-methylguanosine | G |
| oxo8G | 8-hydroxyguanosine | G |
| I | inosine | I |
| Psi | pseudouridine | P |
| Q | Queuosine | Q |
| m3Um | 3,2′-O-dimethyluridine | U |
| mem5s2U | 5-methoxycarbonylmethyl-2- | U |

According to the embodiments of a method according to the invention, an epitranscriptomic profile can include, depending on the requirements of the application, a larger number of modified nucleosides, to be determined from among the known nucleosides (Jonkhout et al, “The RNA modification landscape in human disease”, RNA, December; 23 (12): 1754-1769, 2017). A complete list of all the modified nucleosides which, depending on the requirements of the analysis, may be included in the transcriptomic profiles is publicly accessible.

The epitranscriptomic profile of a sample characterizes said sample. Said epitranscriptomic profile can be obtained by any known technique of the state of the art, and in particular by mass spectrometry, in particular by mass spectrometry coupled with chromatography.

To denote the items of medical information to be predicted, the terms “clinical variables” or “clinical characteristics” will also be used.

In a method according to the invention, the step of analysis of the epitranscriptomic profile for the purposes of clinical prediction is based on a supervised machine learning method. Learning is performed on the profiles originating from a cohort, i.e. cell samples for which the characteristic clinical variable to be predicted is known beforehand. The “computational model” thus created by learning can then be used (in prediction mode) so as to predict the clinical variable for any new sample.

The inventors have also developed a method for normalization of the raw quantitative data making it possible to obtain an epitranscriptomic profile containing comparable relative quantities.

Prior to the learning process, exploratory analysis of the profiles of the samples of the cohort revealed variations in the profiles, said variations being correlated with the grade of the tumours of the subjects from whom the samples were extracted. Based on the profiles of the signature cohort and by means of a machine learning prediction tool, a method according to the invention makes it possible to predict the grade of a glioma based on a biological sample from a patient suffering from a tumour, in particular based on a sample comprising tumour cells. More particularly, a method according to the invention makes it possible to distinguish grades II and III of a glioma based on a sample comprising tumour cells.

Finally, by combining the normalized epitranscriptomic profiles and the patient survival data, and by means of a machine learning prediction tool, a method according to the invention makes it possible to predict the survival of a patient based on a biological sample isolated from said patient, in particular based on a tumour sample.

The inventors have also developed a method for detecting the presence of a tumour in an individual, based on a biological sample isolated from this individual, comprising the steps of:
a) isolating nucleosides from said biological sample, by extracting: i) total cellular RNA and its nucleoside fragmentation, ii) extracellular RNA and its nucleoside fragmentation, and/or iii) nucleosides originating from the monomeric catabolites,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of the presence of said tumour.

The technical steps of a method for detecting the presence of a tumour in an individual match the same characteristics as the technical steps of a method for characterizing a tumour.

Thus the inventors have now developed a method that makes use of the quantitative data of the epitranscriptome for characterizing a tumour, on the one hand, and for detecting the presence of a tumour, on the other hand. According to an embodiment, a method according to the invention is thus advantageously used for characterizing a tumour. According to another embodiment, a method according to the invention is advantageously used for detecting the presence of a tumour.

### DETAILED DESCRIPTION OF THE INVENTION

According to a first aspect, the invention relates to an in vitro method for characterizing a tumour of an individual, based on a biological sample isolated from this individual, comprising the steps of:
a) isolating nucleosides from said biological sample, by extracting: i) total cellular RNA and its nucleoside fragmentation, ii) extracellular RNA and its nucleoside fragmentation, and/or iii) nucleosides originating from the monomeric catabolites,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of said tumour.

According to a first embodiment, a method according to the invention is based on the simultaneous analysis of the quantity of different nucleosides originating from total cellular RNA of a biological sample, and/or originating from extracellular RNA and its nucleoside fragmentation and/or originating from nucleosides obtained from the monomeric catabolites present in said sample, a method according to the invention thus comprises the simultaneous analysis of multiple variables, and not on the quantitative detection of a single marker.

By “profile” or “nucleoside profile” is meant a vector of quantities of nucleosides.

By “total cellular RNA” is meant the totality of cellular RNA extracted according to the well-known and accessible methods. Total cellular RNA includes transfer RNA (tRNA), messenger RNA (mRNA), ribosomal RNA (rRNA) and other non-coding RNAs. Said total cellular RNA is thus present here in a polymeric form.

By “extracellular RNA” is meant the totality of extracellular RNA present in polymeric form, extracted according to the well-known and accessible methods. This polymeric form of extracellular RNA is in particular also denoted by the expression “circulating RNA”. Said extracellular RNA originates from the in vivo enzymatic degradation of transport RNA (tRNA), messenger RNA (mRNA) and/or ribosomal RNA (rRNA) and the other types of RNA, in particular the non-coding RNAs.

By “nucleosides originating from the monomeric catabolites” is meant the nucleosides obtained, according to the well-known and accessible methods, from the catabolites present in a monomeric form in the sample. These monomeric catabolites originate from the in vivo enzymatic degradation of transport RNA (tRNA), messenger RNA (mRNA) and/or ribosomal RNA (rRNA) and the other types of RNA, in particular the non-coding RNAs.

By “isolating and determining a respective quantity of at least 3 different nucleosides” is meant isolating and determining a quantity of each of the “at least 3” nucleosides taken individually.

According to this first embodiment, the invention thus relates to an in vitro method for characterizing a tumour of an individual, based on a biological sample isolated from this individual, comprising the steps of:
a) isolating nucleosides from said biological sample by extracting total cellular RNA and its nucleoside fragmentation,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of said tumour.

The nucleosides can also be present in the biological sample in an extracellular polymeric form, in particular also denoted by the expression “circulating RNA”. The nucleosides can also be present in a monomeric form (metabolites) in the biological sample. Said extracellular RNAs and monomeric nucleosides originate from the in vivo enzymatic degradation of transport RNA (tRNA), messenger RNA (mRNA) and/or ribosomal RNA (rRNA) and the other types of RNA, in particular the non-coding RNAs.

According to a second embodiment, a method according to the invention is based on the simultaneous analysis of the quantity of different nucleosides originating from extracellular RNA of a biological sample.

According to this second embodiment, the invention relates to an in vitro method for characterizing a tumour of an individual, based on a biological sample isolated from this individual, comprising the steps of:
a) isolating nucleosides from said biological sample, by extracting extracellular RNA and its nucleoside fragmentation,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of said tumour.

According to a third embodiment, a method according to the invention is based on the simultaneous analysis of the quantity of different nucleosides originating from the monomeric catabolites present in a biological sample.

According to this third embodiment, the invention relates to an in vitro method for characterizing a tumour of an individual, based on a biological sample isolated from this individual, comprising the steps of:
a) isolating nucleosides from said biological sample, by extracting monomeric catabolites present in said sample,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of said tumour.

In an in vitro method for characterizing a tumour of an individual, based on a biological sample isolated from said individual, said biological sample is in particular selected from:
a solid biological sample, particularly a biopsy, and more particularly a biopsy of said tumour, and
a liquid biological sample, in particular a bodily fluid taken from said individual, more particularly a blood, plasma, serum or urine sample.

By “biopsy” is meant sampling a very small part of an organ or of a tissue. When the biological sample is a biopsy, the first embodiment of the method according to the invention, in which total cellular RNA is extracted then fragmented, is preferred.

When the biological sample is a liquid biological sample, the second and the third embodiment of the method according to the invention, in which extracellular RNA is extracted then fragmented, or in which RNA in the form of isolated nucleosides is extracted, respectively, are preferred.

In a method according to the invention, said biological sample is in sufficient volume, or includes a sufficient number of cells, to allow a reliable quantitative determination of at least 3 nucleosides originating from the fragmentation of an extract of total cellular RNA from said sample.

In the case of a biopsy, total cellular RNA is extracted according to a method selected from the methods accessible to a person skilled in the art, in particular a method such as described in the present example. In the case of a liquid sample, such as blood or urine, said sample is treated beforehand if necessary, so as in particular to eliminate any interfering compounds, to concentrate said sample and/or to determine a standard concentration value of a reference element, such as creatinine in urine, this standard value serving to standardize the concentration of the sample based on which the nucleoside profile is established.

In a method according to the invention, the total cellular RNA, extracellular RNA and isolated nucleosides are obtained from a biological sample by any method known to a person skilled in the art; said method comprises in particular an extraction step, optionally a fragmentation step, and a dephosphorylation step.

According to a particular aspect, the invention thus relates to an in vitro method for characterizing a tumour of an individual based on a biopsy of said tumour, said method comprising preparing, based on said biopsy, an extract of total cellular RNA and nucleoside fragmentation of said RNA.

According to an embodiment, in an in vitro method for characterizing a tumour of an individual according to the invention, at least 3 isolated nucleosides originating from the biological sample, obtained i) by preparing an extract of total cellular RNA and its nucleoside fragmentation, ii) by preparing an extract of extracellular RNA and its nucleoside fragmentation, and/or iii) by extracting the isolated nucleosides, are isolated and their respective quantity determined; said at least 3 nucleosides are selected from:
the unmodified nucleosides: adenosine (A), cytidine (C), guanosine (G), uridine (U), and
the modified nucleosides (see Table 2).

The modified nucleosides result from the action of a large number of highly specific enzymes; the nucleosides undergo in particular methylation and rearrangement of carbon-nitrogen bonds. Said modified nucleosides are all modified nucleosides known at the date of the present application; these nucleosides are in particular mentioned in the publication by Jonkhout et al (“The RNA modification landscape in human disease”, RNA, December; 23 (12): 1754-1769, 2017), and in Table 2 of the present application.

According to different embodiments of a method to which the invention relates, said at least 3 nucleosides are selected from the groups constituted by:
the unmodified nucleosides: adenosine (A), cytidine (C), guanosine (G), uridine (U),
2′-O-methyladenosine (Am), 1-methyladenosine (m1A), N6,N6-dimethyladenosine (m66A), N6,N6,2′-O-trimethyladenosine (m66Am), N6-methyladenosine (m6A), N6,2′-O-dimethyladenosine (m6Am), N4-acetylcytidine (ac4C), 2′-O-methylcytidine (Cm), 5-hydroxymethylcytidine (hm5C), 3-methylcytidine (m3C), 5-methylcytidine (m5C), 2′-O-methylguanosine (Gm), 1-methylguanosine (m1G), N2,N2,7-trimethylguanosine (m227G), N2,7-dimethylguanosine (m27G), 7-methylguanosine (m7G), 8-hydroxyguanosine (oxo8G), inosine (I), pseudouridine (Psi), queuosine (Q), 3,2′-O-dimethyluridine (m3Um), 5-methoxycarbonylmethyl-2-thiouridine (mcm5s2U), 5-methoxycarbonylmethyluridine (mcm5U), 5-carbamoylmethyluridine (ncm5U), 2′-O-methyluridine (Um), and/or 3-(3-amino-3-carboxypropyl) uridine (acp3U), 2′-O-ribosyladenosine (phosphat) (Ar(p)), 5-carboxymethylaminomethyl-2-thiouridine (cmnm5s2U), 5-carboxymethylaminomethyluridine (cmnm5U), 5-carboxymethylaminomethyl-2′-O-methyluridine (cmnm5Um), dihydrouridine (D), 5-formylcytidin (f5C), galactosyl-queuosine (galQ), 2′-O-methyl-5-hydroxymethylcytidine (hm5Cm), 5-hydroxyuridine (ho5U), 5-hydroxyadenosine (ho8A), 8-hydroxyguanosine (ho8G), N6-isopentenyladenosine (i6A), N6-(cis-hydroxyisopentenyl) adenosine (io6A), 1-methylinosine (m1I), 1-methylpseudouridine (m1psi), N2,N2-dimethylguanosine (m22G), 2-methyladenosine (m2A), N2-methylguanosine (m2G), 5-methyluridine (m5U), 5,2′-O-dimethyluridine (m5Um), N6-methyl-N6-threonylcarbamoyladenosine (m6t6A), mannosyl-queuosine (manQ), 5-(carboxyhydroxymethyl) uridine methyl ester (mchm5U), 5-methylaminomethyl-2-thiouridine (mnm5s2U), 2-methylthio-N6-isopentenyladenosine (ms2i6A), 2-methylthio-N6-threonylcarbamoyladenosine (ms2t6A), peroxywybutosine (o2yW), 2′-O-methylpseudouridine (psi m), 2-thiouridine (s2U), N6-threonylcarbamoyladenosine (t6A), wybutosine (yW).

According to an embodiment of a method to which the invention relates, said at least 3 nucleosides are selected from the groups constituted by:
the unmodified nucleosides: adenosine (A), cytidine (C), guanosine (G), uridine (U), and
2′-O-methyladenosine (Am), 1-methyladenosine (m1A), N6,N6-dimethyladenosine (m66A), N6,N6,2′-O-trimethyladenosine (m66Am), N6-methyladenosine (m6A), N6,2′-O-dimethyladenosine (m6Am), N4-acetylcytidine (ac4C), 2′-O-methylcytidine (Cm), 5-hydroxymethylcytidine (hm5C), 3-methylcytidine (m3C), 5-methylcytidine (m5C), 2′-O-methylguanosine (Gm), 1-methylguanosine (m1G), N2,N2,7-trimethylguanosine (m227G), N2,7-dimethylguanosine (m27G), 7-methylguanosine (m7G), 8-hydroxyguanosine (oxo8G), inosine (I), pseudouridine (Psi), queuosine (Q), 3,2′-O-dimethyluridine (m3Um), 5-methoxycarbonylmethyl-2-thiouridine 5-(mcm5s2U), methoxycarbonylmethyluridine (mcm5U), 5-carbamoylmethyluridine (ncm5U), 2′-O-methyluridine (Um).

According to a more particular embodiment, a method to which the invention relates comprises the isolation and quantitative determination of at least 3, 4, 5, 6, 7, 8, 9, 10, 11, 12, 13, 14, 15, 16, 17, 18, 19, 20, 21, 22, 23, 24, 25, 26, 27, 28 or 29 different nucleosides originating from the fragmentation of total RNA from said biological sample.

According to another more particular embodiment, a method to which the invention relates comprises the isolation and quantitative determination of at least 3, 4, 5, 6, 7, 8, 9, 10, 11, 12, 13, 14, 15, 16, 17, 18, 19, 20, 21, 22, 23, 24, 25, 26, 27, 28 or 29 different nucleosides originating from the fragmentation of extracellular RNA from said biological sample.

According to another more particular embodiment, a method to which the invention relates comprises the isolation and quantitative determination of at least 3, 4, 5, 6, 7, 8, 9, 10, 11, 12, 13, 14, 15, 16, 17, 18, 19, 20, 21, 22, 23, 24, 25, 26, 27, 28 or 29 different nucleosides originating from the extraction of nucleosides from said biological sample.

According to a more particular embodiment, a method to which the invention relates comprises the isolation and quantitative determination of at least 3 different nucleosides originating from the fragmentation of total RNA from said biological sample and/or from the fragmentation of extracellular RNA and/or from the extraction of the isolated nucleosides, said nucleosides being selected from the following: adenosine (A), cytidine (C), guanosine (G), uridine (U), 2′-O-methyladenosine (Am), 1-methyladenosine (m1A), N6,N6-dimethyladenosine (m66A), N6,N6,2′-O-trimethyladenosine (m66Am), N6-methyladenosine (m6A), N6,2′-O-dimethyladenosine (m6Am), N4-acetylcytidine (ac4C), 2′-O-methylcytidine (Cm), 5-hydroxymethylcytidine (hm5C), 3-methylcytidine (m3C), 5-methylcytidine (m5C), 2′-O-methylguanosine (Gm), 1-methylguanosine (m1G), N2,N2,7-trimethylguanosine (m227G), N2,7-dimethylguanosine (m27G), 7-methylguanosine (m7G), 8-hydroxyguanosine (oxo8G), inosine (I), pseudouridine (Psi), queuosine (Q), 3,2′-O-dimethyluridine (m3Um), 5-methoxycarbonylmethyl-2-thiouridine (mcm5s2U), 5-methoxycarbonylmethyluridine (mcm5U), 5-carbamoylmethyluridine (ncm5U), 2′-O-methyluridine (Um).

In a method according to the invention, the isolation and determination of a respective quantity of at least 3 nucleosides are implemented by any means of analysis known to a person skilled in the art. These means comprise in particular chromatography, in particular reversed-phase high-performance liquid chromatography (RP-HPLC) or capillary electrophoresis (CE).

These means also comprise spectrometry means, in particular mass spectrometry. More particularly, these means comprise tandem mass spectrometry coupled with liquid-phase chromatography (LC-MS/MS), an analytical technique that combines the separating power of liquid-phase chromatography with the highly sensitive and selective mass analysis capability of triple-quadrupole mass spectrometry. The strong point of this technique resides in the separating power of liquid-phase chromatography for a wide range of compounds, combined with the capability of mass spectrometry of quantifying the compounds with a high degree of sensitivity and selectivity, as a function of the unique mass/charge (m/z) transitions of each compound of interest.

According to a particular aspect, in a method according to the invention, the mixture of nucleosides obtained by fragmentation is analyzed using a high-performance liquid chromatography coupled with a tandem mass spectrometry (LC-MS/MS) of the triple quadrupole type in multiple reaction monitoring (MRM) mode. MRM mode is a highly sensitive and specific technique that makes it possible to quantify molecules by mass spectrometry. This scan mode is dependent on tandem and more particularly triple-quadrupole mass spectrometry or hybrid ion-trap mass spectrometry systems. MRM scan mode is based on selecting ions of specific mass and charge number of a molecule, ions called precursor ions or parent ions, as well as on the corresponding fragment ions after fragmentation in the collision cell. The first quadrupole will allow precise selection of the specific precursor ions of the molecules of interest that will then be fragmented in the second quadrupole. The resultant fragment ions are then selected in the third quadrupole. The two ions (mass/charge) then correspond to a highly specific transition of the molecule of interest.

According to a more particular embodiment, the invention relates to an in vitro method for characterizing a tumour of an individual, based on a biopsy from this individual, comprising the steps of:
a) preparing, based on said biological sample, an extract of total cellular RNA and nucleoside fragmentation of polymeric RNA,
b) isolating and determining a respective quantity of at least 3, preferably at least 5, preferably at least 10, preferably at least 20, different nucleosides originating from step a),
c) establishing, for said biological sample, a nucleoside profile based on the respective quantities of each of the nucleosides obtained during step b), said profile being characteristic of said tumour.

According to an even more particular embodiment, the invention relates to an in vitro method for characterizing a tumour of an individual, said tumour being a tumour situated in one of the following organs: rectum, colon, breast, pancreas, kidney, lung, or a haematological tumour, in particular a leukaemia.

According to a more particular embodiment, the invention relates to an in vitro method for characterizing a glial tumour of an individual, based on a biological sample isolated from this individual, comprising the steps of:
a) preparing, based on said biological sample, an extract of total cellular RNA and nucleoside fragmentation of said RNA,
b) isolating and determining a respective quantity of at least 3, preferably at least 5, preferably at least 10, preferably at least 20, different nucleosides originating from step a),
c) establishing, for said biological sample, a nucleoside profile based on the respective quantities of each of the nucleosides obtained during step b), said profile being characteristic of said tumour, and
d) predicting a grade of said glial tumour by a first classification model trained beforehand, based on the profile established during step c).

The terms “glial tumour” or “glioma” group together various brain tumours that develop from the normal glial cells of the brain. The grade of a glial tumour represents the most important determinant for the survival of an individual having such a tumour. Non-tumoral brain tissue is characterized by numerous cells having normal characteristics and some mitotic characteristics, without endothelial proliferation. Grade II tumours, also called “astroblastomas” comprise a larger number of cells comprising polymorphic nuclei during mitosis. Grade III tumours are also called “anaplastic astroblastomas”. Grade IV tumours correspond to glioblastoma multiforme.

By “classification model” is meant a machine learning algorithm trained beforehand, in particular during supervised learning, as well as a training dataset making it possible to train the aforementioned algorithm, and an evaluation dataset.

According to embodiments, said first classification model can comprise:
a machine learning algorithm,
more particularly a supervised learning neural network, or
a multi-class probabilistic classification algorithm,
   trained beforehand with a training dataset.

The training data are specific to the question posed, on the one hand, and specific to the type of cancer targeted, on the other hand. Thus, the training phase of the learning algorithm uses the training data to produce a classification model that is itself specific to the question posed and specific to the type of cancer targeted. The learning phase infers the parameters of the model as a function of these data and of the question. For example, for a question of determining the grade, the classification model sends back a response from among four possible responses, if four grades are distinguished. Conversely, for the question of detecting the presence of a tumour, the classification model responds with “tumour” or “healthy”, i.e. a choice from two possible responses.

The classification model produced by the learning phase is a program implemented on a computer so as to obtain a prediction on the question considered, based on the data item of an epitranscriptomic profile originating from a sample. This program can be downloaded and installed, thus allowing installation on a system other than the one on which it was produced.

The training dataset can comprise a multitude of data pairs, each of the data pairs comprising a first data item representing a nucleoside profile and a second data item representing the tumour grade for this profile.

The training dataset can comprise a training set and a test set, also denoted by “evaluation set [CF1]”, of the model. The model can thus be tested on the training set and the test set can be used to determine if the model's learning is satisfactory or not.

The training set and the test set can be different. Alternatively, the test set can correspond to a part of the training set.

The training dataset can be formed beforehand based on data obtained in the laboratory by analysis of samples obtained from individuals suffering from cancer and for whom the grade of the tumour has been determined beforehand.

It is considered that the classification model has reached a satisfactory level of learning on all of the profiles of the test set if the classification reaches for example 85% precision; in other words, it is considered that the classification model has reached a satisfactory level of learning on all of the profiles of the test set if the classification reaches for example 15% error at most.

The classification model can consist of a computer program. According to a preferred embodiment of the invention, a classification model implemented in a method according to the invention consists of a computer program that potentially executes a technical function consisting of steps of the classification method. Execution of said program by a computer produces a digital object, which is a technical object.

Said computer program can be written in any computer language such as for example in C, C++, Java, Python, etc.

According to embodiments, the classification model can comprise a support vector machine, a random forest, a linear discriminant analysis (LDA).

More particularly, said learning algorithm is in particular selected from:
a support vector machine provided either with a linear kernel, or with a radial basis function (RBF) kernel with a low cost parameter value,
an LDA algorithm using least-square solutions with automatic determination of the dimension shrinkage parameter according to the Ledoit-Wolf procedure.

These three families of machine learning algorithms are described conceptually in the literature (Cornuejols and Miclet, “Apprentissage Artificiel: Concepts et Algorithmes” [Machine learning: concepts and algorithms] Eyrolles, 2012; Hastie et al. “The Elements of Statistical Learning: Data Mining, Inference, and Prediction”, 2nd Edition. Springer Series in Statistics, Springer 2009, ISBN 9780387848570) and are perfectly suitable for multi-class classification.

According to embodiments of a method according to the invention, predicting a grade of a glial tumour can comprise:
predicting a grade II glial tumour,
predicting a grade III glial tumour or
predicting a grade IV glial tumour.

More particularly, the invention relates to an in vitro method for predicting a grade of a glial tumour of an individual, based on a biological sample from said individual, and in particular a biopsy of said glial tumour, in which predicting a grade of said glial tumour by a classification model trained beforehand, comprises: predicting a grade II glial tumour, predicting a grade III glial tumour and predicting a grade IV glial tumour. More particularly, a method according to the invention for predicting a grade of a glial tumour of an individual comprises distinguishing between a grade II glial tumour and a grade III or IV glial tumour; distinguishing between a grade III glial tumour and a grade II or IV glial tumour; distinguishing between a grade IV glial tumour and a grade II or III glial tumour.

Even more particularly, the invention relates to an in vitro method for characterizing a glial tumour of an individual, based on a biopsy of said tumour, comprising:
a) preparing, based on said biopsy, an extract of total cellular RNA and nucleoside fragmentation of said RNA,
b) isolating and quantitatively determining at least 3 nucleosides originating from said fragmentation, selected from: adenosine (A), cytidine (C), guanosine (G), uridine (U), 2′-O-methyladenosine (Am), 1-methyladenosine (m1A), N6,N6-dimethyladenosine (m66A), N6,N6,2′-O-trimethyladenosine (m66Am), N6-methyladenosine (m6A), N6,2′-O-dimethyladenosine (m6Am), N4-acetylcytidine (ac4C), 2′-O-methylcytidine (Cm), 5-hydroxymethylcytidine (hm5C), 3-methylcytidine (m3C), 5-methylcytidine (m5C), 2′-O-methylguanosine (Gm), 1-methylguanosine (m1G), N2,N2,7-trimethylguanosine (m227G), N2,7-dimethylguanosine (m27G), 7-methylguanosine (m7G), 8-hydroxyguanosine (oxo8G), inosine (I), pseudouridine (Psi), queuosine (Q), 3,2′-O-dimethyluridine (m3Um), 5-methoxycarbonylmethyl-2-thiouridine (mcm5s2U), 5-methoxycarbonylmethyluridine (mcm5U), 5-carbamoylmethyluridine (ncm5U), 2′-O-methyluridine (Um),
c) establishing, for said tumour, a profile based on the respective quantitative values of the nucleosides obtained during step b), said profile being characteristic of said tumour, and
d) predicting a grade of said glial tumour by a classification model trained beforehand, based on the profile established during step c), in which predicting a grade of a glial tumour is selected from: predicting a grade II glial tumour, predicting a grade III glial tumour and predicting a grade IV glial tumour.

According to another aspect, the invention relates to an in vitro method for characterizing a glial tumour of an individual, said method comprising the steps of:
a) preparing, based on said biological sample, an extract of total cellular RNA and nucleoside fragmentation of said RNA,
b) isolating and determining a respective quantity of at least 3, preferably at least 5, preferably at least 10, preferably at least 20, different nucleosides originating from step a),
c) establishing, for said biological sample, a nucleoside profile based on the respective quantities of each of the nucleosides obtained during step b), said profile being characteristic of said tumour, and
d) predicting a survival status of said individual, by a second classification model trained beforehand, based on the profile established during step c).

According to embodiments, said second classification model can comprise:
a machine learning algorithm,
more particularly a supervised learning neural network, or
a probabilistic classification algorithm, trained beforehand with a second training dataset.

Said second training dataset can comprise a multitude of data pairs, each of the data pairs comprising a first data item representing a nucleoside profile and a second data item representing the survival status for this profile.

This training dataset can comprise a training set and an evaluation set of the model. The model can thus be tested on the training set and the evaluation set can be used to determine if the model's learning is satisfactory or not. The training set and the evaluation set can be different. Alternatively, the evaluation set can correspond to a part of the training set. The training dataset can be formed beforehand based on data obtained in the laboratory by analysis of samples obtained from individuals suffering from cancer and for whom the survival status has been determined beforehand.

It is considered that the classification model has reached a satisfactory level of learning on all of the profiles of the evaluation set if the classification reaches 85% precision; in other words, it is considered that the classification model has reached a satisfactory level of learning on all of the profiles of the evaluation set if the classification reaches 15% error at most.

As for the first classification model, the second classification model can consist of a computer program. The computer program can be written in any computer language such as for example in C, C++, Java, Python, etc.

According to embodiments, the second classification model can comprise a support vector machine, a random forest, a linear discriminant analysis.

According to another aspect, the invention relates to a classification model, trained beforehand on a training dataset, for predicting a grade of a glial tumour of an individual suffering from a tumour, based on a nucleoside profile obtained by implementing a method according to the invention.

Said classification model for predicting the grade of a glial tumour comprises a machine learning algorithm trained and evaluated beforehand, in particular during supervised learning, with a training dataset relating to predicting a grade of a glial tumour, said training set comprising a training set and an evaluation set, both relating to predicting a grade of a glial tumour.

The invention also relates to a method for constructing a classification model for predicting the grade of a glial tumour, comprising at least:
selecting a machine learning algorithm for a classification task,
providing a training dataset relating to predicting a grade of a glial tumour, comprising a training set and a test set,
a learning stage of predicting a grade of a glial tumour by said algorithm, using said training dataset.

According to another particular aspect, the invention relates to a second classification model, trained beforehand on a training dataset, for predicting a survival status of an individual suffering from a tumour, based on a nucleoside profile obtained by implementing a method according to the invention.

Said classification model for predicting a survival status of an individual suffering from a tumour comprises a machine learning algorithm trained and evaluated beforehand, in particular during supervised learning, with a training dataset relating to predicting a survival status of an individual, said training set comprises a training set and a test set, both relating to predicting a survival status of an individual suffering from a tumour.

The invention also relates to a method for constructing a classification model for predicting a survival status of an individual, comprising at least:
selecting a machine learning algorithm for a classification task,
providing a training dataset relating to predicting a survival status of an individual, comprising a training set and a test set,
a learning stage of predicting a survival status of an individual, by said algorithm, using said training dataset.

According to another aspect, the present invention relates to the use of a classification model according to the invention for predicting a grade of a glial tumour.

According to an aspect, the present invention relates to the use of a classification model according to the invention for stratification of a patient suffering from a glial tumour, in combination with at least one other biological marker characteristic of said patient.

According to another aspect, the present invention relates to the use of a classification model according to the invention for predicting a survival status of an individual.

According to another particular embodiment, the invention relates to an in vitro method for detecting the presence of a tumour in an individual, based on a biological sample isolated from this individual, comprising the steps of:
a) isolating nucleosides from said biological sample, by extracting: i) total cellular RNA and its nucleoside fragmentation, ii) extracellular RNA and its nucleoside fragmentation, and/or iii) nucleosides originating from the monomeric catabolites, and preferably nucleosides originating from the monomeric catabolites,
b) isolating and determining a respective quantity of at least 3, preferably at least 5, preferably at least 10, preferably at least 20, different nucleosides obtained during step a), and
c) establishing, for said biological sample, a nucleoside profile based on the respective quantities of each of the nucleosides obtained during step b), said profile being characteristic of the presence of said tumour.

According to an even more particular embodiment, the invention relates to an in vitro method for detecting the presence of a tumour in an individual, based on a blood sample isolated from this individual, comprising the steps of:
a) isolating nucleosides from said biological sample, by extracting nucleosides originating from the monomeric catabolites,
b) isolating and determining a respective quantity of at least 3, preferably at least 5, preferably at least 10, preferably at least 20, different nucleosides originating from step a),
c) establishing, for said biological sample, a nucleoside profile based on the respective quantities of each of the nucleosides obtained during step b), said profile being characteristic of said tumour, and
d) predicting the presence of said tumour by a classification model trained beforehand, based on the profile established during step c).

According to an even more particular embodiment, the invention relates to an in vitro method for detecting the presence of a colorectal tumour in an individual, based on a blood sample isolated from this individual, comprising the steps of:
a. isolating nucleosides from said biological sample, by extracting nucleosides originating from the monomeric catabolites,
b. isolating and determining a respective quantity of at least 3, preferably at least 5, preferably at least 10, preferably at least 20, different nucleosides originating from step a),
c. establishing, for said biological sample, a nucleoside profile based on the respective quantities of each of the nucleosides obtained during step b), said profile being characteristic of said tumour, and
d. predicting the presence of said colorectal tumour by a classification model trained beforehand, based on the profile established during step c).

The invention also relates to the use of a method according to the invention for detecting the presence of a tumour, said tumour being a tumour situated in one of the following organs: rectum, colon, breast, pancreas, kidney, lung, or a haematological tumour, in particular a leukaemia.

The invention also relates to the use of a method according to the invention for detecting the presence of a tumour of the digestive tract, in particular a colorectal tumour.

According to another aspect, the invention relates to a classification model, trained beforehand on a training dataset, for detecting the presence of a tumour in an individual, based on a nucleoside profile obtained by implementing a method according to the invention. This classification model comprises a machine learning algorithm trained and evaluated beforehand, in particular during supervised learning, with a training dataset relating to detecting the presence of a tumour in an individual, said training set comprises a training set and a test set, both relating to detecting the presence of a tumour in an individual.

More particularly, the invention relates to a classification model, trained beforehand on a training dataset, for detecting the presence of a colorectal tumour in an individual, based on a nucleoside profile obtained by implementing a method according to the invention. Said classification model comprises a machine learning algorithm trained and evaluated beforehand, in particular during supervised learning, with a training dataset relating to detecting the presence of a colorectal tumour in an individual.

The invention also relates to a method for constructing said classification model for detecting the presence of a tumour, comprising at least:
selecting a machine learning algorithm for a classification task,
providing a training dataset relating to detecting the presence of a tumour in an individual, comprising a training set and a test set,
a learning stage of predicting the presence of a tumour in an individual, by said algorithm, using said training dataset.

According to a particular embodiment, the invention also relates to a method for constructing a classification model for detecting the presence of a colorectal tumour, comprising at least: selecting a machine learning algorithm for a classification task, providing a training dataset relating to detecting the presence of a colorectal tumour in an individual, comprising a training set and a test set, and a learning stage of predicting the presence of a colorectal tumour in an individual, by said algorithm, using said training dataset.

According to another aspect, the present invention relates to the use of a classification model according to the invention for detecting a tumour, in particular a colorectal tumour.

According to an aspect, the present invention relates to the use of a classification model according to the invention for detecting a tumour, in particular a colorectal tumour, in combination with at least one other biological marker characteristic of said patient.

According to another particular aspect, the present invention finally relates to a diagnostic method comprising implementing a method according to the invention for characterizing a tumour. The present invention also relates to a diagnostic method comprising implementing a method according to the invention for predicting a grade of a glial tumour. The present invention also relates to a diagnostic method comprising implementing a method according to the invention for predicting the survival status of a patient. Said diagnostic method can moreover comprise a histological analysis of the tissues.

According to another particular aspect, the present invention finally relates to a diagnostic method comprising implementing a method according to the invention for detecting a tumour. The present invention also relates to a diagnostic method comprising implementing a method according to the invention for detecting a colorectal tumour. Said diagnostic method can comprise a histological analysis of the tissues.

### DESCRIPTION OF THE FIGURES AND EMBODIMENTS

Other advantages and characteristics will become apparent on examination of the detailed description of an embodiment that is in no way limitative, and from the attached drawings, in which:

FIG. 1 shows the overall experimental scheme, where LC-MS/MS denotes liquid chromatography combined with mass spectrometry and the raw data (data) are the epitranscriptomic profiles obtained by LC-MS/MS.

FIG. 2 shows the overall scheme of the bioinformatic process, the raw data are the epitranscriptomic profiles obtained by LC-MS/MS, the normalized data are the epitranscriptomic profiles after normalization, MS denotes mass spectrometry (combined with liquid chromatography).

FIGS. 3A, 3B and 3C show, in the form of a box-and-whisker plot, six graphs respectively showing the relative quantity (as a percentage) of six modified nucleosides according to the grade of glial tumour. For each of the graphs, said grade is denoted on the x-axis by: “Normal”, “Grade-II”, “Grade-III” or “Grade-IV” respectively indicating a sample of glial tissue that is non tumoral or a sample of glial tumour of grade II, III or IV. FIG. 3A shows two examples of nucleosides the quantity of which decreases with the increasing grade of the glial tumour: (from left to right) oxo8G and m1G. FIG. 3B shows two examples of nucleosides the quantity of which increases with the increasing grade of the glial tumour: (from left to right) m6Am and Gm. FIG. 3C shows two examples of nucleosides the quantity of which varies slightly with the increasing grade of the glial tumour: (from left to right) m1A and m7G. The scales are different according to the graphs.

FIG. 4 shows the explained variance percentage of the first components of the principal component analysis (PCA) of the epitranscriptomic profiles of the cohort. On the x-axis, the components are numbered from 0 to 9. On the y-axis are the explained variance percentages for these components.

FIG. 5 shows the visualization in three dimensions of the profiles of the cohort according to said first three components of the principal component analysis (PCA), i.e. the three components that represent 39.2+23.3+8.6=71.1% of the variance of the epitranscriptomic profiles of the cohort. Each of the axes shows, respectively, the principal component 0 (39.24%), the principal component 1 (23.27%) and the principal component 2 (8.58%). The “star” symbols represent the “normal” grade; the “triangle”, grade II; “square”, grade III and “cross”, grade IV, respectively.

It is well understood that the embodiments that will be described hereinafter are in no way limitative. Variants of the invention can be envisaged in particular comprising only a selection of characteristics described hereinafter, in isolation from the other characteristics described, if this selection of characteristics is sufficient to confer a technical advantage or to differentiate the invention with respect to the state of the prior art. The present invention will be better understood on reading the following example, which is given to illustrate the invention and not to limit the scope thereof.

### EXAMPLE 1: ANALYSIS OF THE TRANSCRIPTOMIC DATA OF SAMPLES OF GLIAL CELLS

This section presents the cohort used, the preparation of the samples, the method for obtaining epitranscriptomic profiles, and the computer analysis program. This section then presents the results of exploratory analysis of the profiles of the cohort, prediction of the grades of the tumours and prediction of survival.

Preparation of the samples and obtaining the profiles by mass spectrometry are carried out as follows: fifty-eight samples originating from surgically resected tumours in adult patients diagnosed with a glioma, none of the patients having received chemotherapy or radiotherapy before surgery, were used in accordance with the French bioethics laws with respect to patient information and consent. At the time of resection, for each tumour, an aliquot was immediately frozen and stored at −80° C. and the remaining tissue was fixed in 4% formalin, incorporated in paraffin and sections of 3 microns were cut and stained with haematoxylin and eosin. The histopathological type of the tumour was determined according to the revised World Health Organization classification (Wesseling & Capper, “WHO 2016 Classification of gliomas”. Neuropathol Appl Neurobiol. 44, 139-150, 2018). The group of tumours is constituted by grade II (n=20), grade III (n=20) gliomas and grade IV (n=18) glioblastomas. Moreover, 19 “control” samples of non-tumoral glial cells (n=19) were prepared according to the same protocol (described hereinafter) as the tumour samples.

Total RNA was extracted from samples of tumours by using the acid guanidinium-phenol method. The quality of the RNA samples was determined by agarose gel electrophoresis and staining with ethidium bromide, and the 18S and 28S RNA bands were visualized under UV light. Treatment of the biological sample starts by extracting RNA by phase separation so as to obtain an RNA sample of at least 100 ng. Treatment continues with enzymatic hydrolysis of polymeric RNA and dephosphorylation of the nucleosides.

Enzymatic digestion of the RNA is carried out as follows: a quantity of 400 ng RNA is diluted in a total volume of 20 μL milliQ water to which are added 3 μl ammonium acetate (0.1 M pH 5.3) and 0.001 enzyme unit (U) Nuclease P1 (Sigma, N8630).

Incubation at 42° C. is carried out for 2 hours. Then, 3 μl ammonium acetate 1 M and 0.001 U alkaline phosphatase (Sigma, P4252) are added. The mixture is then incubated at 37° C. for 2 hours. Finally, the nucleoside solution is diluted twice and filtered with 0.22 μm filters (Millex®-GV, Millipore, SLGVR04NL). Finally, 5 μL of each sample is injected and all the samples are analyzed in triplicate by LC-MSMS.

Liquid chromatography (LC) is carried out as follows: the nucleosides are separated by Nexera LC-40 systems (Shimadzu), using a Synergi™ Fusion-RP C18 column (particle size 4 μm, 250 mm×2 mm, 80 Â) (Phenomenex, 00G-4424-B0). The mobile phase is constituted by ammonium acetate 5 mM adjusted to pH 5.3 with acetic acid (solvent A) and pure acetonitrile (solvent B). Gradient elution for 30 minutes starts with 100% phase A followed by a linear gradient to 8% solvent B at 13 minutes. Solvent B is further increased to 40% in 10 minutes. After 2 minutes, solvent B is brought back to 0% at 25.5 minutes. The initial conditions are regenerated by rinsing with 100% solvent A for an additional 4.5 minutes. The flow rate is 0.4 ml/min and the column temperature is 35° C.

Mass spectrometry in multiple reaction monitoring (MRM) mode is carried out as follows: detection is carried out by Shimadzu TripleQuad 8060 in positive-ion mode. The mass spectrometry operates in dynamic MRM mode with a retention time window of 3 min and a maximum cycle time set at 258 ms. The areas of the peaks are determined using the Skyline 4.1 software (Pino L K et al, “The Skyline ecosystem: Informatics for quantitative mass spectrometry proteomics.” Mass Spectrom Rev. 2020 May; 39 (3): 229-244. 2020).

The mass spectrometer was calibrated to identify and quantify with precision 25 modified nucleosides (Table 2) and 4 unmodified nucleosides (A, U, G, T) (Table 1). The mass spectrometry appliance used is a Shimadzu TripleQuad 8060 in multiple reaction monitoring mode.

Each sample was injected three times, thus providing three technical replicates. For each nucleoside, the homogeneity of the retention time given by the mass spectrometer is verified. Measurements showing a divergence greater than 6% were discarded. A data table results therefrom containing the quantity measurements of each nucleoside, in each replicate, for all the samples. This table is then analyzed by virtue of the applicants' computer programs.

All the bioinformatic analyses are carried out with internally developed Python programs. To this end, the authors used well-known open source modules: “Pandas” for the management of tabular data (Reback et al, Pandas-dev/pandas: Pandas 1.0.3 (Version v1.0.3). Zenodo Mar. 18, 2020), “scikit-learn” for the exploratory statistical analyses of the data and for machine learning (Pedregosa et al, Journal of Machine Learning Research, vol. 12, pp. 2825-2830, 2011), “Matplotlib” for the visualization (JD Hunter, Computing in Science & Engineering, vol. 9, no. 3, pp. 90-95, 2007).

The necessary characteristics of the programs are the following: a) they accept as input mass spectrometry data in a file in tabular form (.csv format); b) the quantifications of area and retention time originating from the spectrometer must be given in the form of real values with a precision of at least 10-1; c) they implement a multi-class supervised machine learning algorithm from among those mentioned above; d) they implement the learning phase, the validation phase, and the prediction mode; e) they use the classification model in prediction mode to classify the epitranscriptome profile of a patient sample so as to predict the grade of the tumour.

For the data pre-processing and the normalization, the raw quantities table is loaded into memory and its format is verified. Then, the average quantity of each nucleoside is calculated, and the table is reformatted to obtain all the measurements in one row for each biological sample. Mass spectroscopy does not produce absolute calculations of molecules but relative measurements. The inventors propose a new normalization formula, in which the quantities of the unmodified nucleosides A, C, G and U are added together. This sum serves as a reference. Then all the reference measurements are divided by this sum. Thus, relative measurements are obtained, all comprised within the interval [0, 1]. By way of example, an extract from such a data table is given in Tables 3, 4 and 5.

TABLE 3

| Grade | A | Am | C | Cm | G |
|---|---|---|---|---|---|
| Grade-II | 1.455E−01 | 5.507E−02 | 5.185E−01 | 1.219E−02 | 3.258E−01 |
| Grade-III | 7.084E−02 | 7.919E−02 | 4.303E−01 | 1.670E−02 | 4.915E−01 |
| Grade-IV | 1.360E−01 | 7.150E−02 | 3.681E−01 | 1.776E−02 | 4.894E−01 |
| Normal | 1.599E−01 | 6.030E−02 | 5.014E−01 | 1.406E−02 | 3.286E−01 |

| Grade | Gm | I | Psi | Queuosine | U |
|---|---|---|---|---|---|
| Grade-II | 7.194E−03 | 1.774E−04 | 4.391E−03 | 5.045E−05 | 1.021E−02 |
| Grade-III | 1.006E−02 | 1.632E−04 | 4.090E−03 | 3.430E−05 | 7.414E−03 |
| Grade-IV | 9.499E−03 | 2.988E−04′ | 3.922E−03 | 2.500E−05 | 6.571E−03 |
| Normal | 7.311E−03 | 1.992E−04 | 2.928E−03 | 9.334E−05 | 1.007E−02 |

TABLE 4

| Grade | Um | ac4C | hm5C | m1A | m1G |
|---|---|---|---|---|---|
| Grade-II | 9.863E−05 | 1.255E−04 | 2.075E−06 | 3.333E−02 | 1.073E−03 |
| Grade-III | 1.489E−04 | 8.378E−05 | 3.249E−07 | 2.740E−02 | 3.793E−04 |
| Grade-IV | 1.760E−04 | 1.112E−04 | 1.660E−06 | 5.166E−02 | 6.559E−04 |
| Normal | 1.246E−04 | 1.780E−04 | 3.208E−06 | 4.849E−02 | 1.914E−03 |

| Grade | m227G | m27G | m3C | m3Um | m5C |
|---|---|---|---|---|---|
| Grade-II | 1.300E−04 | 3.744E−06 | 3.017E−03 | 2.522E−06 | 2.728E−02 |
| Grade-III | 2.149E−04 | 3.739E−06 | 2.587E−03 | 4.434E−06 | 2.273E−02 |
| Grade-IV | 1.593E−04 | 6.544E−06 | 5.398E−03 | 5.382E−06 | 4.159E−02 |
| Normal | 1.553E−04 | 9.019E−06 | 4.470E−03 | 1.799E−06 | 3.654E−02 |

TABLE 5

| Grade | m66A | m66Am | m6A | m6Am | m7G | mcm5U | mcm5s2U | ncm5U | oxo8G |
|---|---|---|---|---|---|---|---|---|---|
| Grade-II | 3.035E−03 | 1.746E−06 | 2.785E−03 | 1.303E−04 | 6.834E−03 | 1.599E−06 | 2.199E−05 | 3.996E−05 | 2.454E−06 |
| Grade-III | 4.077E−03 | 8.600E−07 | 4.115E−03 | 2.538E−04 | 6.673E−03 | 9.258E−07 | 3.282E−05 | 1.757E−05 | 2.357E−07 |
| Grade-IV | 3.899E−03 | 9.457E−07 | 4.110E−03 | 3.981E−04 | 1.185E−02 | 2.162E−06 | 7.748E−05 | 7.354E−05 | 1.060E−06 |
| Normal | 3.368E−03 | 6.777E−07 | 3.183E−03 | 1.283E−04 | 9.999E−03 | 8.795E−06 | 1.118E−05 | 5.544E−05 | 5.862E−06 |

Tables 3, 4 and 5 indicate, for each of the nucleosides analyzed, the normalized data value for each of the grades II, III and IV of glioma, and for the healthy tissues (“normal”).

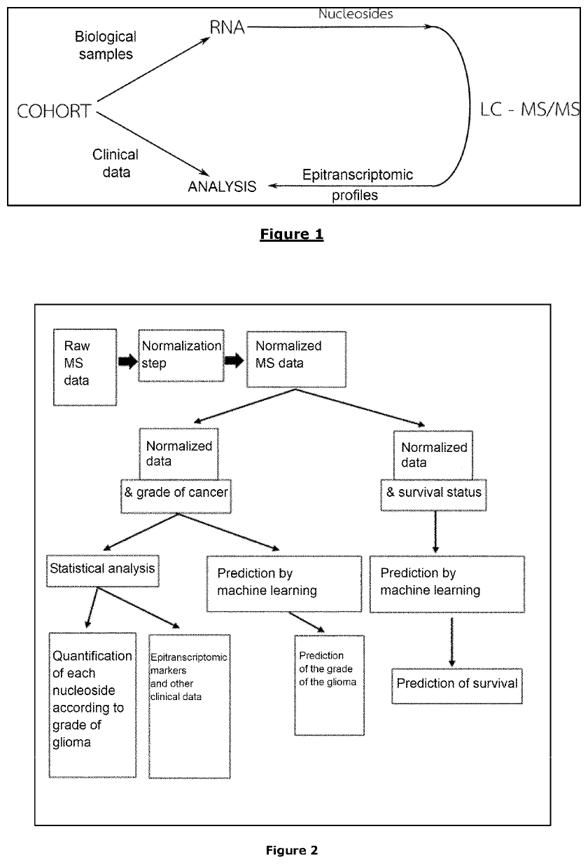

Joint analysis of the epitranscriptomic profiles and of clinical variables of interest (FIG. 1) is carried out, in particular relating to the grade in the case of the gliomas. This procedure can be adapted to any type of clinical variable. In this example, it is sought to distinguish the grades of cancer, which can be difficult to establish by means of anatomopathological examination.

Pre-processing of the profiles of the cohort resulted in a table of 77 rows, with one row per sample, and 29 columns, with one column per measurement. For each of the samples, the legend of the grades of the tumours or the legend “normal” for the healthy samples was added. An exploratory statistical analysis of this table was carried out to evaluate the relevance of the signal contained in the profiles to the grade information.

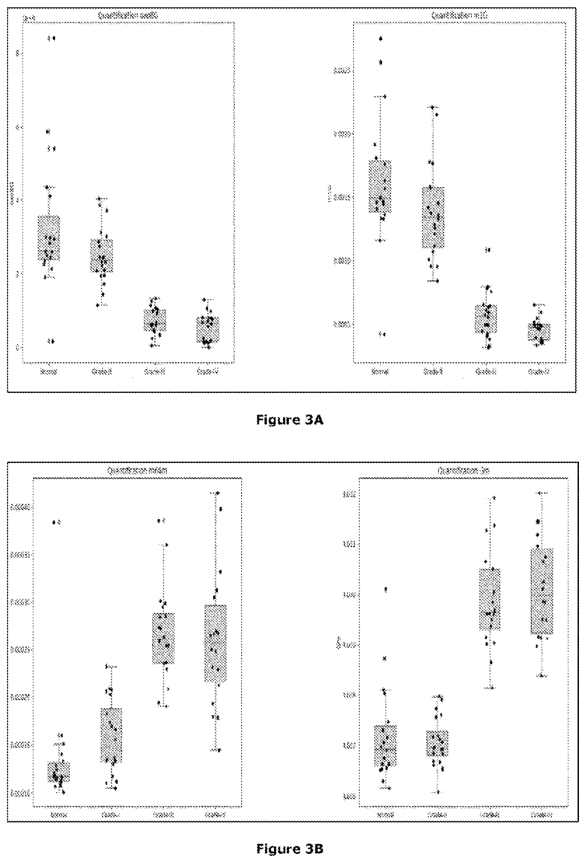

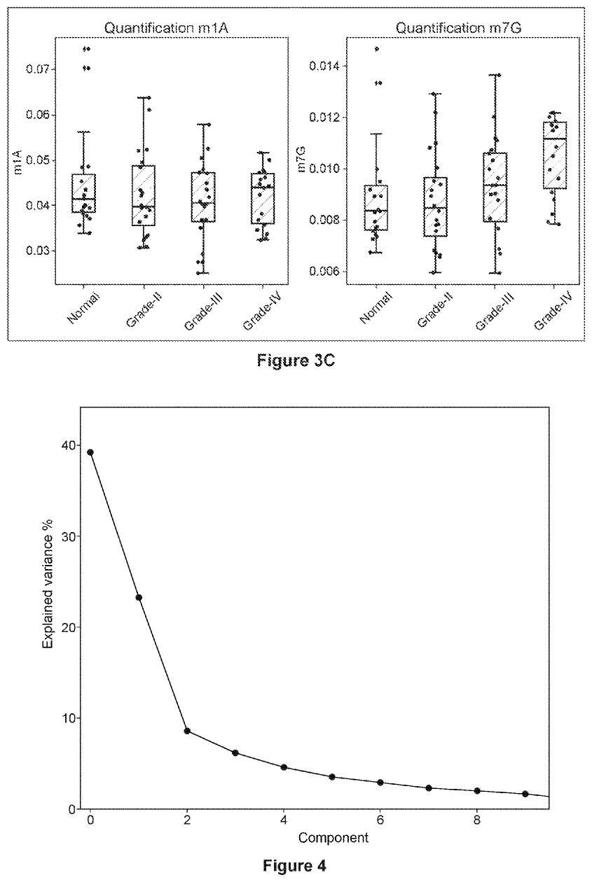

Firstly, the variations in the quantities of each nucleoside are studied in the samples of one and the same grade, and these variations are compared between the grades. As shown in the box-and-whisker-plot graphs in FIGS. 3A, 3B and 3C, the experimental results suggest a grouping together of the nucleosides in four groups: i) those the quantity of which increases with the grade, i.e. between the non-tumoral brain tissue (denoted for the sake of simplicity as “normal” on the y-axis of the graphs) and the grades II, III and IV, in particular the nucleosides oxo8G, m1G, queuosine and Ac4C (as shown for example in FIG. 3A); ii) those the quantity of which decreases with the grade (as shown for example in FIG. 3B); iii) those which vary slightly with the grades (as shown for example in FIG. 3C) and iv) the remaining nucleosides, which do not meet the conditions of belonging to the first three groups.

At first sight, none of these groups is associated with a known specific characteristic of its constituents (for example, modified edge of the nucleoside). However, it should be noted that the 2′-O-methylations (Am, Um, Cm, Gm), mainly found in ribosomal RNA (rRNA) and small nuclear RNA (snRNA), behave similarly in a central cluster containing m6Am, a specific modification of rRNA.

Then, a principal component analysis (PCA) of these data was carried out, so as to perform a dimension reduction, not to be confused with a selection of the “characteristics”, in other words of the nucleosides, to see if the quantity variations could be combined in a small number of components. FIG. 4 shows the explained variance percentage for the first 10 components of the PCA: clearly the first three components group together a large majority of the profile variations. It is noted in fact that the first three components alone group together: 39.2+23.3+8.6=71.1% of the variance of the epitranscriptomic profiles of the cohort.

Each epitranscriptomic profile comprising the measurements for x nucleosides is seen in mathematical terms as a point in a space having x dimensions. PCA is a multivariate exploratory analysis method that makes it possible to reduce the dimensions of the data while still capturing their variability. The components are new variables that combine the data of the initial observations so as to better capture their variability while still reducing the number of variables to be analyzed. The components result from projecting the initial data on other axes of the multidimensional space. The components are ordered in decreasing order of explained variance percentage. This percentage associated with each component indicates its importance for describing the initial data. FIG. 4 shows the graph of the explained variance percentage for the first 10 components. PCA is a standard data analysis technique.

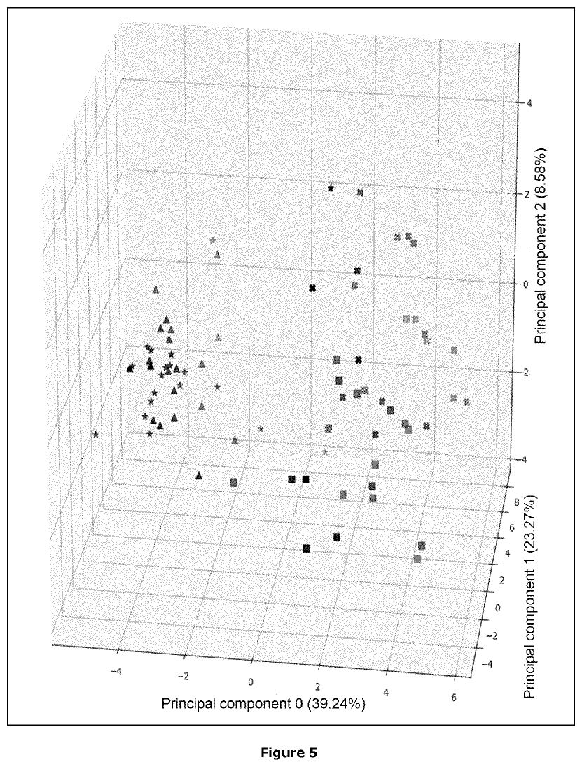

The visualization in 3 dimensions of the projected profiles on the first three components is shown in FIG. 5. Firstly, the samples of non-tumoral tissue and of grade II are clearly separated from those of grades III and IV. Furthermore, the samples of grade III occupy a volume relatively separated from those of grade IV. These exploratory results suggest that supervised machine learning algorithms should be able to learn a boundary between the groups of samples of different grades.

Machine Learning Method Making it Possible to Predict the Grade of the Tumours and the Healthy Samples with Precision

A machine learning method was tested to determine if the grade of the samples could be predicted based only on the epitranscriptomic profiles, i.e. without using any other information than the quantities of nucleosides (FIG. 1). To do this, the profiles were partitioned into two distinct subsets: the first was used only to train the machine learning model (n=60, i.e. 78%), the second served to evaluate the model (n=17, i.e. 22%).

As the variable to be predicted (here the grade) is a categorical data item, the learning method must belong to the classification category. A support vector machine (SVM) classification algorithm was selected from the major types of learning algorithms, for the possibility it offers to adapt the boundary formulas by changing the type of kernel, as is standard in learning. The prediction precision of the SVM algorithm provided with a linear kernel on the profiles of the test subset is 0.90, out of a maximum of 1, which is remarkable. The level of prediction precision is maintained when the learning, then the tests, is reiterated, with new random partitionings of the dataset, which demonstrates the robustness of the learning tool developed.

Moreover, the results of the evaluation make it possible to compare the applicants' normalization method (denoted by SUM, for sum) with the formulas used in the literature. In fact, the conventional normalization that consists of dividing the measurement of a modified nucleoside, for example m1A, by that of the corresponding unmodified nucleoside, here the measurement of A. In Table 6, the precision depending on the use of different formulas is comprised between 0.8 and 0.9, and is therefore always less than or equal to (but never greater than) the precision of the normalization formula SUM.

TABLE 6

| Normalization | A | C | G | U | SUM |
|---|---|---|---|---|---|
| Precision of the | 0.85 | 0.90 | 0.85 | 0.80 | 0.90 |
| prediction | | | | | |

Moreover, the prediction of the grades is robust to change of the classification algorithm. Instead of an SVM algorithm, if an algorithm based on a linear discriminant analysis approach is used, a precision of 92% is obtained, with a recall (or sensitivity) of 90% and an F1 score of 90%. The detail of the predictions for each grade is given in Table 7.

TABLE 7

| Grade | precision | recall | f1 score |
|---|---|---|---|
| Normal | 1.00 | 0.80 | 0.89 |
| Grade-II | 0.67 | 1.00 | 0.80 |
| Grade-III | 1.00 | 0.83 | 0.91 |
| Grade-IV | 0.88 | 1.00 | 0.93 |
| Weighted average | 0.92 | 0.90 | 0.90 |

In conclusion, the quality of prediction of the grades is not particularly associated with the optimization of a learning method on a given cohort, since two very different learning methods obtain similar results. The quality of the prediction is therefore associated with the power of the signal contained in the transcriptomic profiles.

Moreover, the training models the results of which are reported here were intentionally not optimized with respect to their parameters, so as to avoid a risk of overtraining, which would adversely affect the generalization ability of the models.

### Predicting the Patient Survival Status

The same approach by supervised learning was used to predict the clinical variable indicating the survival status, i.e. the “living” or “deceased” status at the end of follow-up of the cohort, i.e. in 2020. Here, the classification is binary: “living” or “deceased”. The SVM learning algorithm gives a correct prediction at 80%, which is plausible with regard to the size of the cohort in question (Table 8).

TABLE 8

| Class | precision | recall | f1 score |
|---|---|---|---|
| False (living) | 0.75 | 0.86 | 0.80 |
| True (deceased) | 0.89 | 0.80 | 0.84 |
| Weighted average | 0.83 | 0.82 | 0.82 |

### Conclusion

Differences in relative quantities of certain epigenetic modifications of the RNAs were highlighted according to different samples, whether they are healthy or tumoral. These differences make it possible in particular to separate the different tumour grades. A supervised machine learning algorithm applied to the vectors of nucleoside quantities makes it possible to efficiently distinguish the grades of the gliomas, and makes it possible in particular to distinguish grades II and III, with a remarkable precision taking account of the relatively limited size of the cohort. Furthermore, this method also makes it possible, based on the same data, to estimate the survival of the patients using a supervised machine learning method.

### EXAMPLE 2: ANALYSIS OF THE TRANSCRIPTOMIC DATA OF BLOOD SERUM SAMPLES FROM SUBJECTS

Forty-seven blood samples from adult patients diagnosed with a colorectal cancer or from control subjects (Etablissement Français du Sang [French blood service], n=20), none of the patients having received chemotherapy or radiotherapy before surgery, were used in accordance with the French bioethics laws with respect to patient information and consent. A local ethics committee (Comité de Recherche Translationnelle [translational research committee] (CORT)) assessed and authorized the use of these samples.

Circulating RNA is extracted from plasma, using a kit (miRNeasy Serum/Plasma). The RNAs are digested with nuclease P1 and treated with alkaline phosphatase so as to obtain a mixture of nucleosides. The circulating free nucleosides are extracted from the same plasma samples, using an extraction procedure by methanol. They do not require enzyme treatment before passing to mass spectrometry.

The liquid chromatography (LC) and the mass spectrometry and the bioinformatic analyses are carried out as indicated in Example 1. In particular, each sample is analyzed three times independently, thus making it possible to obtain three technical replicates for each. The raw data are processed so as to be normalized as in Example 1. Thus, these steps produce one epitranscriptomic profile per sample. A machine learning method was developed and tested to determine the presence or absence of a tumour based only on the epitranscriptomic profiles, i.e. without using any other information than the quantities of nucleosides. As in Example 1, a support vector machine (SVM) classification algorithm provided with a linear kernel was selected for this binary task called “classification” task. The algorithm was first trained and then tested so as to evaluate its ability to predict the presence or absence of a tumour in the sample. With the epitranscriptomic profiles containing the measurements of the free nucleosides, the machine learning method gives a prediction with a precision of 100% and a sensitivity of 100%.

1. An in vitro method for characterizing a tumour of an individual, based on a biological sample isolated from this individual, comprising the steps of:
a) isolating nucleosides from said biological sample, by extracting: i) total cellular RNA and its nucleoside fragmentation, ii) extracellular RNA and its nucleoside fragmentation and/or iii) nucleosides originating from the monomeric catabolites;
b) isolating by chromatography and determining a respective quantity of at least 3, different nucleosides originating from step a); and
c) establishing, for said biological sample, a nucleoside profile based on the respective quantities of each of the nucleosides obtained during step b), said profile being characteristic of said tumour.

2. The method according to claim 1, in which said biological sample is a biopsy or a liquid biological sample, said liquid biological sample being selected from blood, serum, plasma and urine.

3. The method according to claim 1, in which said nucleosides are selected from the following:
the unmodified nucleosides: adenosine (A), cytidine (C), guanosine (G), uridine (U); and
the modified nucleosides: 2′-O-methyladenosine (Am), 1-methyladenosine (m1A), N6,N6-dimethyladenosine (m66A), N6,N6,2′-O-trimethyladenosine (m66Am), N6-methyladenosine (m6A), N6,2′-O-dimethyladenosine (m6Am), N4-acetylcytidine (ac4C), 2′-O-methylcytidine (Cm), 5-hydroxymethylcytidine (hm5C), 3-methylcytidine (m3C), 5-methylcytidine (m5C), 2′-O-methylguanosine (Gm), 1-methylguanosine (m1G), N2,N2,7-trimethylguanosine (m227G), N2,7-dimethylguanosine (m27G), 7-methylguanosine (m7G), 8-hydroxyguanosine (oxo8G), inosine (I), pseudouridine (Psi), queuosine (Q), 3,2′-O-dimethyluridine (m3Um), 5-methoxycarbonylmethyl-2-thiouridine (mcm5s2U), 5-methoxycarbonylmethyluridine (mcm5U), 5-carbamoylmethyluridine (ncm5U), 2′-O-methyluridine (Um).

4. The method according to claim 1, characterized in that said tumour is a glial tumour and in that it comprises a step of predicting a grade of said glial tumour by a first classification model trained beforehand, based on the profile established during step c.

5. The method according to claim 4, characterized in that the first classification model comprises:
a machine learning algorithm,
a supervised learning neural network, or
a multi-class probabilistic classification algorithm,
trained beforehand with a training dataset.

6. The method according to claim 4, in which predicting a grade of a glial tumour is selected from: predicting a grade II glial tumour, predicting a grade III glial tumour and predicting a grade IV glial tumour.

7. The method according to claim 1, characterized in that it comprises a step of predicting a survival status of said individual, by a second classification model trained beforehand, based on the profile established during step c).

8. The method according to claim 7, characterized in that the second classification model comprises:
a machine learning algorithm,
a supervised learning neural network, or
a probabilistic classification algorithm,
trained beforehand with a training dataset.

9. An in vitro method for detecting the presence of a tumour in an individual, based on a biological sample isolated from said individual, comprising the steps of:
a) isolating nucleosides from said biological sample, by extracting: i) total cellular RNA and its nucleoside fragmentation, ii) extracellular RNA and its nucleoside fragmentation and/or iii) nucleosides originating from the monomeric catabolites;
b) isolating by chromatography and determining a respective quantity of at least 3, different nucleosides originating from step a); and
c) establishing, for said biological sample, a nucleoside profile based on the respective quantities of each of the nucleosides obtained during step b), said profile being characteristic of the presence of said tumour.

10. A classification model, trained beforehand on a training dataset, for predicting, in a method according to claim 4,
a grade of a tumour, and/or
a survival status of an individual,
based on the profile established during step c).

11. A use of a classification model according to claim 9, for predicting a grade of a tumour and/or for predicting a survival status of an individual.

12. A use of a method according to claim 1 for detecting a tumour.

13. The use according to claim 12 for detecting a colorectal tumour.
